(19) United States
(12) Patent Application Publication
(10) Pub. No.: US 2023/0223099 A1
(43) Pub. Date: Jul. 13, 2023

(54) PREDICTING METHOD OF CELL DECONVOLUTION BASED ON A CONVOLUTIONAL NEURAL NETWORK

(71) Applicant: Shanghai Institute of Technology, Shanghai (CN)

(72) Inventors: Zhendong Liu, Shanghai (CN); Xinrong Lv, Shandong (CN); Yunxiang Liu, Shanghai (CN); Ying Chen, Shanghai (CN)

(73) Assignee: Shanghai Institute of Technology, Shanghai (CN)

(21) Appl. No.: 18/150,201

(22) Filed: Jan. 5, 2023

(30) Foreign Application Priority Data
Jan. 5, 2022 (CN) 202210003514.7

(51) Int. Cl. G16B 5/00; G16B 30/00; G16B 40/20; G06N 3/08

(52) U.S. Cl. CPC G16B 5/00; G06N 3/08; G16B 30/00; G16B 40/20

(57) ABSTRACT

A predicting method of cell deconvolution based on a convolutional neural network is provided. The convolutional neural network technology is used to speculate the cell type composition proportion of a tissue from single-cell RNA sequencing data. Compared with a traditional cell deconvolution algorithm, the predicting method of cell deconvolution based on a convolutional neural network overcomes the defects that the traditional cell deconvolution algorithm needs to carry out complex data preprocessing and needs to design a mathematical algorithm to standardize the single-cell sequencing data. According to the convolutional neural network designed by the present disclosure, hidden features can be extracted from the single-cell RNA sequencing data, network nodes have very high robustness to noise and errors of the data, and internal relations among various genes are fully mined, so that the cell deconvolution performance is improved. Meanwhile, the model of the present disclosure is established based on the neural network.

### CROSS-REFERENCE TO RELATED APPLICATION

This application claims the priority benefit of China application no. 202210003514.7, filed on Jan. 5, 2022. The entirety of the above-mentioned patent application is hereby incorporated by reference and made a part of this specification.

### BACKGROUND

### Technical Field

The present disclosure mainly relates to the field of downstream analysis based on single-cell RNA sequencing data, and mainly relates to a cell deconvolution method, in particular to a cell deconvolution method for single-cell RNA sequencing data based on a convolutional neural network.

### Description of Related Art

With the wide application of high-throughput sequencing technology in the fields of biology and medicine, the single-cell RNA sequencing technology developed in recent years can perform unbiased, repeatable, high-resolution and high-throughput transcription analysis on a single cell. The traditional sequencing technology performs sequencing based on population cells, which reflects the average expression value of a group of cells, but cannot reveal the heterogeneity among different cells. However, the single-cell RNA sequencing technology can study the expression profile of a single cell, so as to prevent the gene expression value of a single cell from being masked by the average value of the population, and reveal the heterogeneity of complex cell populations. The single-cell RNA sequencing technology extracts, reversely transcribes, amplifies and sequences all RNA of a single cell to obtain single-cell RNA sequencing data. The analysis of the sequencing data can reveal the cell composition of biological tissues, discover rare cell groups, and explore the changes of cell components.

Cell deconvolution is an aspect of downstream analysis of single-cell RNA sequencing data. Cell deconvolution infers the cell type and proportion of the tissue from the single-cell RNA sequencing data of tissue samples, which can be used to discover new cell subtypes, discuss the immune infiltration of cancer tissues, explore the pathogenesis of diseases, etc. However, the traditional deconvolution algorithm has some drawbacks. For example, the used mathematical model needs to add various constraints to standardize the model, and the model is not intuitive enough and is unreadable. Complicated data preprocessing is required, and the accuracy of gene expression matrix of a specific cell type and the accuracy of gene expression matrix of a tissue are high. At present, machine learning technology is not widely used in the field of cell deconvolution. There is still much room for exploration in using machine learning technology to improve the performance of cell deconvolution. In order to solve these problems, a new cell deconvolution scheme urgently needs to be developed to meet the higher demands of biomedical data processing and analysis.

### SUMMARY

Aiming at the defects of the existing cell deconvolution algorithm, the present disclosure provides a predicting method Cbccon of cell deconvolution based on a convolutional neural network. Cbccon predicts the proportion of tissue cells by using deep learning technology, that is, convolutional neural network. The hidden nodes of a Cbccon model can effectively mine the internal relations among genes. The nodes can learn the features of robustness to noise and deviation, which has better deconvolution performance. The purpose of establishing the Cbccon model is to solve the problems that the current cell deconvolution algorithm is affected by noise and deviation so as to result in low accuracy and various constraints need to be added to standardize the model.

In order to achieve the above purpose, the present disclosure provides the following technical scheme. A method of cell deconvolution based on a convolutional neural network is provided, including the following steps:
(1) using single-cell RNA sequencing data to simulate artificial tissues, and determining the total number K of cells in a simulated artificial tissue and the number Q of artificial tissues to be generated; extracting K cells from the single-cell RNA sequencing data, and combining a gene expression matrix of the extracted cells to form a gene expression matrix of the simulated artificial tissue X = {X₁,X₂,..,X₁..,Xₙ}, in which X₁ (≤1≤1≤n) is the feature of the simulated tissue, and denoting the proportion Z = {Z_{1,}Z₂,..,Zᵢ,..Zₜ} (1 ≤ i ≤ t) of each cell type in the tissue as the marking information of the tissue, in which Zᵢ (1 ≤ i ≤ t) is the cell proportion of a certain cell type in the tissue; t is the number of cell types in the tissue; K is a positive integer greater than 1, and Q is a positive integer greater than 1;
(2) screening the features of the simulated artificial tissue X = {X_{1,}X₂,.., Xᵢ..,Xₙ},X₁ (1 ≤ 1 ≤ n) obtained in step (1), and converting each feature Xᵢ(1≤i≤n) into logarithmic space and performing normalizing operation on each feature; obtaining a data set X′ through the above processing;
(3) if the data set X′ obtained in step (2) comes from s different data sets, dividing the data set X′ into a training set X′ₜᵣₐᵢₙ and a test set X′ₜₑₛₜ for s-fold cross-validation, in which the training set consists of s-1 data from different sources, and the test set consists of partial data from the remaining one source, determining the batch size, and randomly extracting the batch size data X′_{batch} from the training set X′ₜᵣₐᵢₙ as input data of one training;
(4) obtaining the cell type number t of the tissue from the input data in step (3) as the number of neurons in the last layer of the fully connected module of the convolutional neural network, constructing a convolutional neural network model Cbccon, and determining the learning rate of the model, the testing number of times step of the model training, and the optimized algorithm of the model; inputting X′_{batch} in step (3) as the data of one training into the Cbccon model for performing model training, and obtaining the predicted tissue cell proportion Ẑ = {Ẑ₁,Ẑ₂,..,Ẑᵢ..,Ẑₜ} , in which Ẑᵢ (1≤i≤t) is the cell proportion of a certain cell type in the tissue predicted by the training set; calculating the loss function between the predicted value and the real value of the cell proportion by the formula
$${J}_{M S E} = \frac{1}{\text{t}} {\sum {}_{i = 1}^{i = t} \left({Z}_{i} - {\hat{Z}}_{i}\right)}^{2} ,$$
in which Zᵢ is the real cell fraction label of the tissue, and Ẑᵢ is the cell proportion finely predicted by the tissue of the training set, optimizing the loss function J_{MSE} using the optimized algorithm; according to the step (3), randomly extracting X′_{batch} for step-1 times for continuous training, and after the training, saving the trained parameters in the Cbccon model;
(5) using the Cbccon model trained in step (4) to predict the data, and inputting X′ₜₑₛₜ into the trained model to obtain the prediction result, that is, the predicted tissue cell type proportion Z′ = {Z′₁, Z′₂_{,}..,Zᵢ′..,Z’ₜ} of the test set, in which Zᵢ′ (1≤i≤t) is the cell proportion of a certain cell type in the tissue predicted in the test set data.

The evaluation indexes are constructed by the models obtained in step (4) and step (5), and the performance of the model is evaluated. The performance of a Cbccon model is evaluated by the formula

$$R M S E \left(\text{z} , {\text{z}}^{′}\right) = \sqrt{\text{avg} {\left(\text{z} - {\text{z}}^{′}\right)}^{2}} ,$$

the formula

$$\text{relate} \left(\text{z} , {\text{z}}^{′}\right) = \frac{\text{cov} \left(\text{z} , {\text{z}}^{′}\right)}{{\partial }_{\text{z}} {\partial }_{{\text{z}}^{′}}} ,$$

the formula

$${\text{hrelate(z,z′) = relate(z,z′)}}^{2}$$

respectively, and the

$$\text{uniform (z,z′) =} \frac{2 {\partial }_{z} {\partial }_{z ′} \times \text{relate(z,z′)}}{{\partial }_{z}^{2} {\partial }_{z ′}^{2} + ( {γ}_{z} - {γ}_{z ′} )} ,$$

performance is compared with CPM, Cibersort(Ci), Cibersortx(Cix), and MuSic methods. Z′ is the predicted cell proportion, Z is the actual cell proportion, ∂_{z}, ∂_{z′} represent the standard deviation of the predicted cell proportion and the actual cell proportion, respectively, and γ_{z}, γ_{z}_{′} represent the average of the predicted cell proportion and the actual cell proportion, respectively. By comparing the evaluation indexes of the model, it can be concluded that compared with other algorithms, Cbccon model has a lower RMSE value, a smaller variation range and a higher relate value. This shows that Cbccon method has better deconvolution performance than other algorithms. The improvement of Cbccon on prediction accuracy of cell deconvolution is mainly due to the fact that the convolution layer used in the model can fully mine the internal relations among genes from single-cell RNA sequencing data, thus extracting the hidden features of the data. Moreover, the network nodes of Cbccon have high robustness to the noise and deviation of the data, so that the prediction accuracy of the cell proportion is higher. Moreover, Cbccon solves the problem that the traditional algorithm needs gene expression matrix of a specific cell type to deconvolution the cells, or needs to add various constraints to standardize the model. The model structure is intuitive and understandable, and has high expansibility.

Preferably, in step (1), K is 100-5000, and Q is 1000-100000.

Preferably, using single-cell RNA sequencing data for simulation in step (1) includes the following steps:
(1-1) determining the proportion of each cell type in a single simulated cell tissue by the formula
$${\text{Z}}_{i} = \frac{{\text{f}}_{\text{i}}}{\sum {}_{i = 1}^{i = t} {\text{f}}_{\text{i}}}$$
(≤ i ≤ t), that is, determining the marking information Z = {Z_{1,}Z₂,...,Zᵢ,..Zₜ} of the simulated tissue, in which Zᵢ(1 ≤ i ≤ t) is the cell proportion of a certain cell type in the simulated tissue; fᵢ is a random number created for a single cell type, Zᵢ has a value between [0,1], and
$${\sum }_{\text{i=1}}^{\text{i=t}} {\text{f}}_{\text{i}}$$
is the sum of random numbers created for all cell types, in which
$$\sum {}_{i = 1}^{i = t} {\text{Z}}_{\text{i}} = 1 ;$$
;
(1-2) determining the number of cells of each cell type to be actually extracted for a single simulated cell tissue by the formula Cᵢ = Zᵢ * K (1≤i≤t), that is, determining the number of cells C={C₁,C₂,...,Cᵢ,.,Cₜ} extracted for each cell type of a single simulated cell tissue, in which Cᵢ(1≤i≤t) is the number of cells to be extracted for a single cell type of a simulated tissue, is the cell proportion of a certain cell type in the simulated tissue, K is the total number of cells in a set simulated artificial tissue, and Cᵢ is the number of cells of each cell type to be actually be extracted for a single simulated cell tissue,in which
$${\sum }_{\text{i=1}}^{\text{i=t}} {\text{C}}_{\text{i}} = K .$$

Preferably, the data preprocessing of the simulated artificial tissue X in step (2) includes the following steps:
(2-1) converting Xᵢ(1≤i≤n) data into logarithmic space by the formula
$${\tilde{X}}_{i j} = {log}_{2} ( {X}_{i j} + 1 )$$
to obtain X̃;
(2-2) performing linear normalization on X̃ by the formula
$${X}_{i , n o r m a l}^{′} = \frac{{\tilde{X}}_{i j} - min ( {x}_{i} )}{{\tilde{X}}_{i j} - max ( {x}_{i} )}$$
(1≤i≤n,1≤j≤m) to obtain X′.

Preferably, the value of the batch size in step (3) is 128.

Preferably, in step (4), the Cbccon model is a convolutional neural network which consists of a plurality of the convolution layers, a plurality of the pool layers and a full connection layer, two filter convolution layers with 64 extracted features are used, one maximum pool layer is used to reduce the number of features, two filter convolution layers with 32 extracted features are used, one maximum pool layer is used to reduce the number of features, two filter convolution layers with 16 extracted features are used, one maximum pool layer is used to reduce the number of features, two filter convolution layers with 8 extracted features are used, one maximum pool layer is used to reduce the number of features, two filter convolution layers with 4 extracted features are used, one maximum pool layer is used to reduce the number of features, and then the data is input into a flattening layer to convert the data into one-dimensional data; finally, three full connection layers are used, in which the number of nodes is 128, 64, and the number of cell types, respectively; all convolution layers are one-dimensional, the activation function of the convolution layer is uniformly set as relu function with a step size of 1, the first two full connection layers use the relu activation function, and the last full connection layer uses the softmax layer to predict the proportion of tissue cells.

Preferably, in step (4), the value of the learning rate of the Cbccon model is 0.0001, the value of the testing number of times step of the model training is 5000, and the optimized algorithm of the model is set as RMSprop algorithm.

Compared with the prior art method, the beneficial effects of the present disclosure are as follows.

This patent puts forward a new scheme of cell deconvolution prediction algorithm, which can predict the cell proportion of tissues more accurately. The algorithm simulates gene expression matrix of heterogeneous tissues based on single-cell RNA sequencing data, which solves the problem of expensive acquisition of single-cell RNA sequencing data to a certain extent. Moreover, the method is based on a convolutional neural network. The model structure is clear and understandable, no complicated data preprocessing is required, and no specific cell expression matrix is required to establish a complicated mathematical model.

### BRIEF DESCRIPTION OF THE DRAWINGS

FIG. 1 is a schematic diagram of a model structure of Cbccon.

FIG. 2 shows specific parameters of a Cbccon model.

FIG. 3 shows partial prediction results of a Cbccon test set.

FIG. 4 is a comparison diagram of various evaluation indexes between a Cbccon model and CPM, Cibersort(Ci), Cibersortx(Cix) and MuSic deconvolution models.

FIG. 5 is a comparison diagram of RMSE evaluation indexes between a Cbccon model and CPM, Cibersort(Ci), Cibersortx(Cix) and MuSic deconvolution models.

FIG. 6 is a comparison diagram of relate evaluation indexes between a Cbccon model and CPM, Cibersort(Ci), Cibersortx(Cix) and MuSic deconvolution models.

### DESCRIPTION OF THE EMBODIMENTS

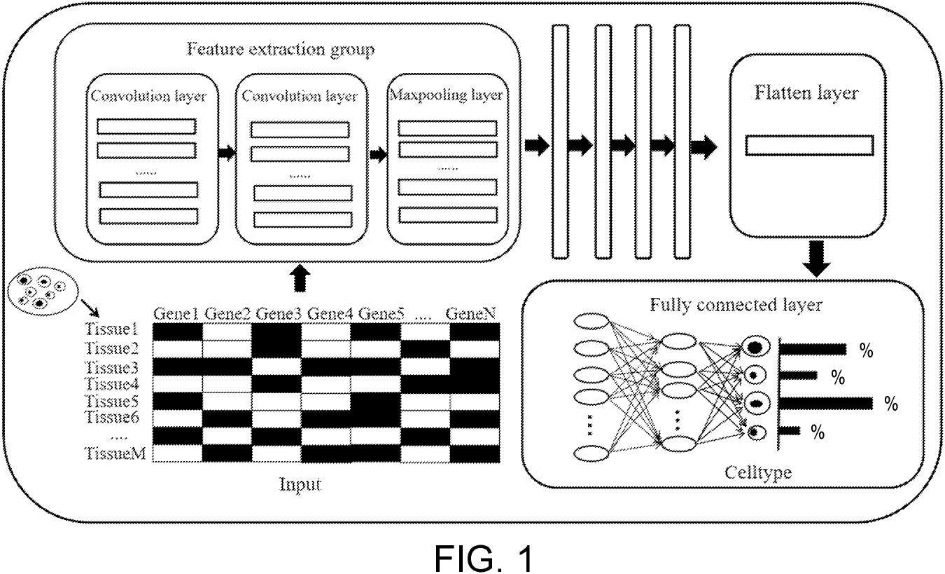

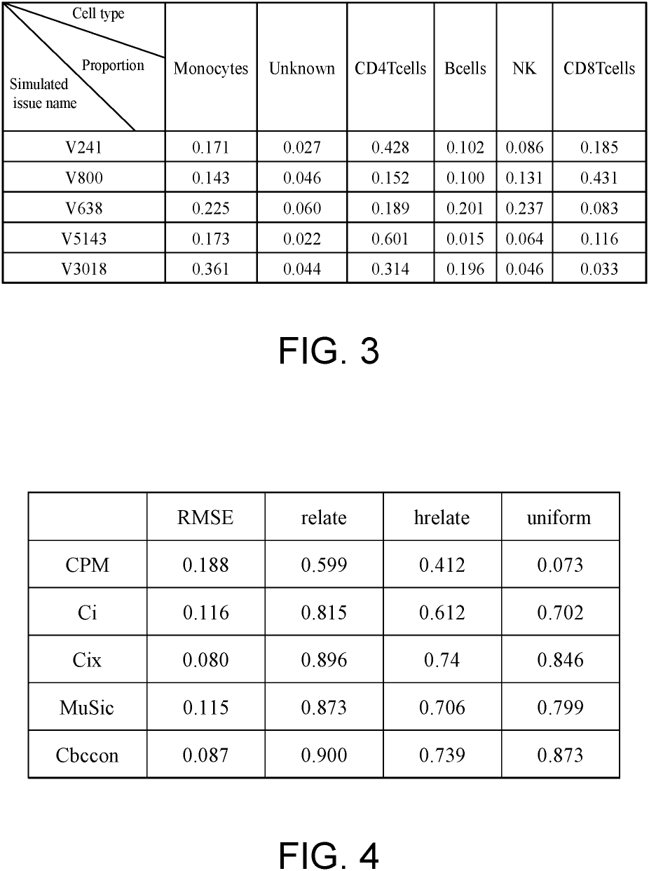

In order to clearly illustrate the technical scheme of the present disclosure, the present disclosure will be described hereinafter with reference to FIGS. 1-6 and examples. The examples here are only used to explain the present disclosure, rather than limit the present disclosure.

It should be pointed out that the following detailed description is exemplary and is intended to provide further explanation of the present disclosure. Unless otherwise indicated, all technical and scientific terms used herein have the same meanings as commonly understood by those skilled in the art to which the present disclosure belongs.

FIG. 1 shows a brief illustration of a Cbccon model for deconvolution of tissue cells using single-cell RNA sequencing data. First, the gene expression moments of the pretreated simulated tissues are input into the convolutional neural network. Each line is the expression amount of each gene of a simulated tissue, and the label of this line is the cell type proportion of the corresponding simulated tissue. The Cbccon model is divided into inputting data into a feature extraction layer, takes two convolution layers and one maximum pool layer as feature extraction layers, performs feature extraction for five times, then inputs the obtained data into the flattening layer, and converts the data format into a one-dimensional vector. Finally, the one-dimensional vector is input into a three-layer fully connected neural network, and the predicted tissue cell proportion can be obtained after training.

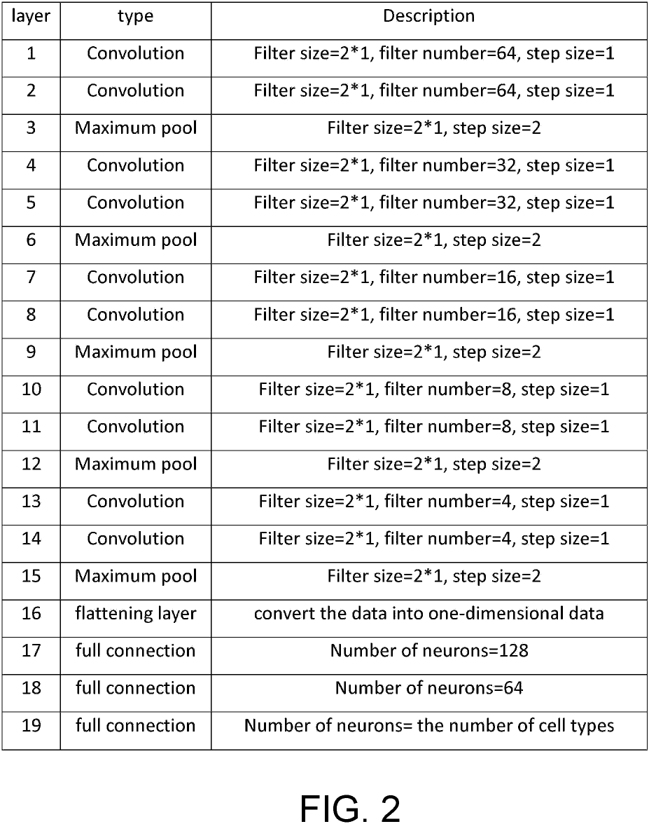

FIG. 2 shows the parameter settings in convolutional neural network. For the first feature extraction layer, two filter convolution layers with 64 extracted features are used, and one maximum pool layer is used to reduce the number of features. Two filter convolution layers with 32 extracted features are used, and one maximum pool layer is used to reduce the number of features. Two filter convolution layers with 16 extracted features are used, and one maximum pool layer is used to reduce the number of features. Two filter convolution layers with 8 extracted features are used, and one maximum pool layer is used to reduce the number of features. Two filter convolution layers with 4 extracted features are used, and one maximum pool layer is used to reduce the number of features. The data is then input into a flattening layer to convert the data into one-dimensional data. Finally, three full connection layers are used, in which the number of nodes is 128, 64, and the number of cell types, respectively. All convolution layers are one-dimensional. The activation function of the convolution layer is uniformly set as relu function with a step size of 1. The first two full connection layers use the relu activation function, and the last full connection layer uses the softmax layer to predict the proportion of tissue cells.

The data is the single-cell RNA sequencing data from human peripheral blood mononuclear cells (PBMC), which comes from four data sets. The above data is cited in the form of data6k, data8k, donorA and donorC herein. The input file of Cbccon contains two txt files, in which the single-cell gene expression matrix of PBMC data is in count.txt, and the type of cells contained in pbmc tissues is in celltype.txt. The output file of Cbccon contains a pb file, a txt file and a csv file. The parameters in the model after training are saved in savemodel.pb file. The prediction.txt predicts the proportion of each cell type in the tissue. The compare.csv file compares the scores of a Cbccon model with various evaluation indexes RMSE, relate, hrelate and uniform of CPM, Ci, Cix and Music methods, so as to compare the performance of the model. The total number of cells in a simulated artificial tissue is set as K=500, and the number of artificial tissues to be generated is set as Q=32000. The number of data in one training is batch size=128. The learning rate of the model is learning rate=0.0001. The testing number of times of the model training is step=5000. The optimized algorithm of the model is set as RMSprop algorithm. The following are the specific steps of performing the cell deconvolution algorithm.

### 1 Single-Cell RNA Sequencing Data Is Used to Simulate Artificial Tissue

Single-cell RNA sequencing data of data6k, data8k, donorA and donorC of PBMC is used to simulate artificial tissues, and the total number K=500 of cells in a simulated artificial tissue and the number Q=32,000 of artificial tissues to be generated are determined. 500 cells are extracted from the single-cell RNA sequencing data, and a gene expression matrix of the extracted cells are combined to form a gene expression matrix of the simulated artificial tissue X = {X₁,X₂,...,Xᵢ,.,Xₙ},Xᵢ(1≤i≤32738), X₀(1≤j≤3200) , which is the feature of the simulated tissue. The proportion Z = {Z_{1,}Z₂,..,Z_{i,}..Zₜ} of each cell type in the tissue is denoted as the marking information of the tissue. Zi(1≤i≤6) is the cell proportion of a certain cell type in the tissue, including the following steps:
(1-1) determining the proportion of each cell type in a single simulated cell tissue by the formula
$${Z}_{i} = \frac{{\text{f}}_{i}}{\sum {}_{i = 1}^{i = 6} {\text{f}}_{i}} ,$$
that is, determining the marking information Z = {Z₁, Z₂,..,Z₁} of the simulated tissue, in which Zᵢ (1≤i≤6) is the cell proportion of a certain cell type in the simulated tissue; fᵢ is a random number created for a
$${\sum }_{\text{i=1}}^{\text{i=6}} {f}_{\text{i}}$$
single cell type, Zᵢ has a value between [0,1], and is the sum of random numbers created for all cell types, in which
(1-2) determining the number of cells of each cell type to be actually extracted for a single simulated cell tissue by the formula Cᵢ = Zᵢ*K (1≤i≤6), K=500, that is, determining the number of cells C = {C₁,C₂,.,Cᵢ..,Cₜ} extracted for each cell type of a single simulated cell tissue, in which Cᵢ(1≤i≤6) is the number of cells to be extracted for a single cell type of a simulated tissue, Zᵢ is the cell proportion of a certain cell type in the simulated tissue, K is the total number of cells in a set simulated artificial tissue, and Cᵢ the number of cells of each cell type to be actually be extracted for a single simulated cell tissue, in which

$${\sum }_{\text{i=1}}^{\text{i=6}} {\text{C}}_{\text{i}} = 500 .$$

### 2. Data Preprocessing

The data of the simulated artificial tissue X = {X₁,X_{2,..,}X_{i,..}Xₙ},X₁(1 ≤ i ≤ 32738) , X₀(1≤ j ≤ 32000) obtained in step 1 is pre-processed. Each feature Xᵢ(1≤i≤32738) n the data set X is screened to remove 21,410 feature items, leaving 11,328 features. Thereafter, X is converted into logarithmic space and normalizing operation is performed. The data set X′ is obtained through the above data pre-processing, including the following steps.

(2-1) the data Xᵢ(1≤i≤32738) is converted into logarithmic space by the formula X̃ᵢⱼ = log₂(Xᵢⱼ + 1) to obtain X̃. X̃₁ is taken as an example, that is, the eigenvalues of the A1BG feature are converted from [105.2, 83.5, 55.8, ...] into [6.73, 6.4, 5.82, ...].

(2-2) the linear normalization is performed on X̃ by the formula

$${x}_{i , n o r m a l}^{′} = \frac{{\tilde{x}}_{i j} - min ( {x}_{i} )}{{\tilde{x}}_{i j} - max ( {x}_{i} )}$$

(1≤i≤n,1≤j≤m), and the value of X̃ᵢ is scaled to [0,1] to obtain X′ . X̃₁ is taken as an example, that is, the maximum value of the A1BG feature is 10.54, and the minimum value thereof is 0.53.

### 3. Dividing the Data Set

The data set X′ obtained in step 2 comes from 4 different data sets, namely, data6k, data8k, donorA and donorC. There are six cell types in the data set, namely, Monocytes, Unknown, CD4Tcells, Bcells, NK and CD8Tcells, in which Unknown represents unknown cell type. The X′ₜᵣₐᵢₙ and a test set X′ₜₑₛₜ for 4-fold cross-validation, data set is divided into a training set and a test set for 4-fold cross-validation, in which the training set consists of 3 data from different sources, and the test set consists of partial data from the remaining one source. The data from data6k, data8k, and donorC are selected from X′ as the training set, and data from donorA is used as the test set. For the convenience of testing, only 500 data are extracted from donorA as the test set. The batch size is determined to be 128. 128 data X′_{batch} are randomly extracted from the training set X′ₜᵣₐᵢₙ as the input data of one training.

### 4. Training the Cbccon Model

The cell type number t=6 of the tissue is obtained from the input data in step 3 as the number of neurons in the last layer of the fully connected module of the convolutional neural network. A convolutional neural network model Cbccon is constructed. It is determined that the learning rate of the model is = 0.0001, the testing number of times step of the model training is =5000, and the optimized algorithm of the model is RMSprop algorithm. X′_{batch} in step 3 as the data of one training is input into the Cbccon model for performing model training, so as to obtain the predicted tissue cell proportion Ẑ = {Ẑ₁, Ẑ₂,..,Ẑᵢ..,Ẑₜ} of the training set, in which Ẑᵢ (1≤i≤6) is the cell proportion of a certain cell type in the tissue predicted by the training set. The loss function between the predicted value and the real value of the cell proportion is calculated by the formula

$${J}_{M S E} = \frac{1}{\text{t}} {\sum }_{\text{i=1}}^{\text{i=6}} {\left({Z}_{i} - {\hat{Z}}_{i}\right)}^{2} ,$$

in which Zᵢ is the real cell fraction label of the tissue, and Ẑᵢ is the cell proportion finely predicted by the tissue. The loss function J_{MSE} is optimized using the optimized algorithm RMSprop. According to the step 3, X′_{batch} is randomly extracted for 4,999 times for continuous training, and after the training, the trained parameters in the Cbccon model are saved.

### 5. Using the Trained Model for Prediction

The Cbccon model trained in step 4 is used to predict the data. The test set data X′ₜₑₛₜ , that is, 500 test data in donorA, is input into the trained model to obtain the prediction result, that is, the predicted tissue cell type proportion Z′ = {Z′₁,Z′₂,..,Zᵢ′..,Z’ₜ} of the test set, in which Zᵢ′ which (1≤i≤t) is the cell proportion of a certain cell type in the tissue predicted in the test set data. Taking a simulated tissue named V241 in the test set as an example, the prediction result of the cell proportion of the tissue of V241 is as follows: the cell proportion of Monocytes type is 0.171; the cell proportion of Unknown type is 0.027; the cell proportion of CD4Tcells type is 0.428; the cell proportion of Bcells type is 0.102; the cell proportion of NK type is 0.086; and the cell proportion of CD8Tcells type is 0.185. The partial prediction results of the cell type proportion of 500 simulated tissues are shown in FIG. 4.

### 6. Model Evaluation

The evaluation indexes are constructed by the models obtained in step 4 and step 5, and the performance of the model is evaluated. The performance of a Cbccon model is evaluated by the formula

$$\text{RMSE} \left(\text{z} , {\text{z}}^{′}\right) = \sqrt{\text{avg} {\left(\text{z} - {\text{z}}^{′}\right)}^{2}} ,$$

the formula

$$\text{relate} \left(\text{z} , {\text{z}}^{′}\right) = \frac{\text{cov} \left(\text{z} , {\text{z}}^{′}\right)}{{\partial }_{\text{z}} {\partial }_{{\text{z}}^{′}}}$$

the formula

$${\text{hrelate(z,z′) = relate(z,z′)}}^{2} ,$$

and the formula

$$\text{uniform} \left(z , {z}^{′}\right) = \frac{2 {\partial }_{z} {\partial }_{{z}^{′}} \times \text{relate} \left(z , {z}^{′}\right)}{{\partial }_{z}^{2} + {\partial }_{{z}^{′}}^{2} + \left({γ}_{z} - {γ}_{{z}^{′}}\right)} ,$$

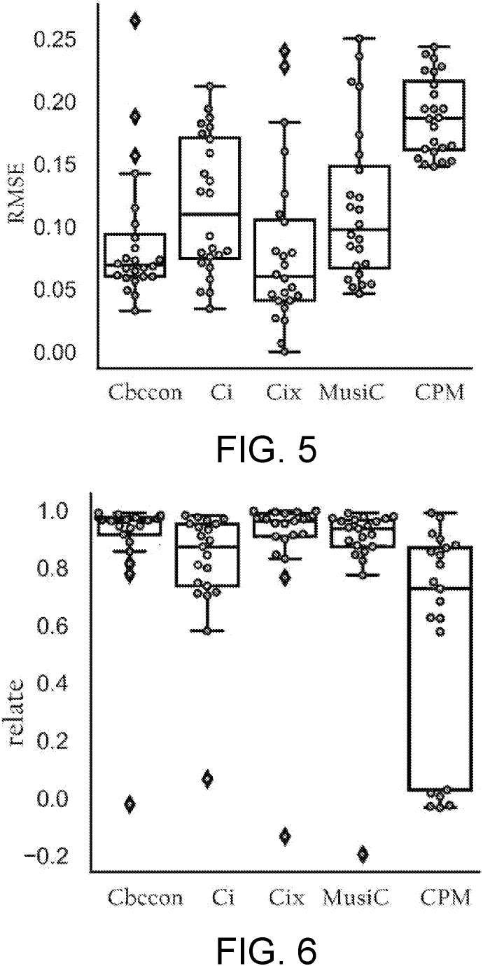

respectively, and the performance is compared with CPM, Cibersort(Ci), Cibersortx(Cix), and MuSic methods. Z′ is the predicted cell proportion, Z is the actual cell proportion, ∂_{z}, ∂_{z′} represent the standard deviation of the predicted cell proportion and the actual cell proportion, respectively, and γ₂, γ₂, represent the average of the predicted cell proportion and the actual cell proportion, respectively. By comparing the evaluation indexes of the model, it can be concluded that compared with other algorithms, Cbccon model has a lower RMSE value, a smaller variation range and a higher relate value. This shows that Cbccon method has better deconvolution performance than other algorithms. The improvement of Cbccon on prediction accuracy of cell deconvolution is mainly due to the fact that the convolution layer used in the model can fully mine the internal relations among genes from single-cell RNA sequencing data, thus extracting the hidden features of the data. Moreover, the network nodes of Cbccon have high robustness to the noise and deviation of the data, so that the prediction accuracy of the cell proportion is higher. Moreover, Cbccon solves the problem that the traditional algorithm needs gene expression matrix of a specific cell type to deconvolution the cells, and needs to add various constraints to standardize the model. The model structure is intuitive and understandable, and has high expansibility. The comparison results are shown in FIG. 4, FIG. 5 and FIG. 6.

After fitting the model with the training data in step 4, the data coverage rate achieved by Cbccon is counted as follows:
(1) data with the error between the predicted value and the true value of the cell proportion within 10%; coverage rate: 99.8%;
(2) data with the error between the predicted value and the true value of the cell proportion within 5%; coverage rate: 85%;
(3) data with the error between the predicted value and the true value of the cell proportion within 1%; coverage: 30%.

Through the comparative result in FIG. 4, FIG. 5 and FIG. 6, it can be seen that the RMSE of Cbccon is lower, and the variation range is smaller. Compared with other methods, the relate correlation is also higher, reaching 0.900, which indicates that the Cbccon model has better accuracy and stronger anti-interference ability to noise in the prediction of the tissue proportion.

Finally, it should be explained that the above is only a preferred embodiment of the present disclosure, and it is not intended to limit the present disclosure. Although the present disclosure has been described in detail with reference to the aforementioned embodiments, it is still possible for those skilled in the art to modify the technical solutions described in the aforementioned embodiments or equivalently replace some of the technical features. Any modification, equivalent substitution, improvement, etc. made within the spirit and principle of the present disclosure shall be included in the scope of protection of the present disclosure.

What is claimed is:

1. A method of cell deconvolution based on a convolutional neural network, comprising the following steps:
(1) using single-cell RNA sequencing data to simulate artificial tissues, and determining a total number K of cells in a simulated artificial tissue and a number Q of artificial tissues that need to be generated; extracting K cells from the single-cell RNA sequencing data, and combining a gene expression matrix of the extracted cells to form a gene expression matrix of the simulated artificial tissue X = {X₁, X₂,.., Xᵤ,..,Xₙ} , in which Xᵤ is a feature of the simulated tissue, 1≤u≤n ; denoting a proportion Z = {Z₁, Z_{2,..}Z_{i,..}Zₜ} of each cell type in the tissue as a marking information of the tissue, in which Zᵢ is the cell proportion of a certain cell type in the tissue, and t is the number of cell types in the tissue, 1≤1≤t; K is a positive integer greater than 1, and Q is a positive integer greater than 1;
(2) screening the features of the simulated artificial tissue X ={X₁, X_{2,..,} X_{u,..,} Xₙ} obtained in step (1), and converting each feature Xᵤ into logarithmic space and performing normalizing operation on each feature, 1 ≤ u ≤ n ; obtaining a data set X′ through the above processing;
(3) if the data set X′ obtained in step (2) comes from s different data sets, dividing the data set X′ into a training set X′ₜᵣₐᵢₙ a test set X′ₜₑₛₜ for s-fold cross-validation, in which the training set consists of s-1 data from different sources, and the test set consists of partial data from the remaining one source, determining the batch size, and randomly extracting the batch size data X′_{batch} from the training set X′ₜᵣₐᵢₙ as input data of one training;
(4) obtaining the cell type number t of the tissue from the input data in step (3) as the number of neurons in the last layer of the fully connected module of the convolutional neural network, constructing a convolutional neural network model Cbccon, and determining the learning rate of the model, the testing number of times step of the model training, and the optimized algorithm of the model; inputting X′_{batch} in step (3) as the data of one training into the Cbccon model for performing model training, and obtaining the predicted tissue cell proportion Ẑ = {Ẑ_{1,}Ẑ₂,.,Ẑᵢ,..,Ẑₜ}, in which Ẑᵢ is the cell proportion of a certain cell type in the tissue predicted by the training set, 1 ≤i ≤ t; calculating the loss function between the predicted value and the real value of the cell proportion by the formula
$${J}_{M S E} = \frac{1}{\text{t}} {\sum }_{\text{i=1}}^{\text{i=t}} {\left({Z}_{\text{i}} - {\dot{Z}}_{\text{i}}\right)}^{2} ,$$
in which Zᵢ is the real cell fraction label of the tissue, and Ẑᵢ is the cell proportion finely predicted by the tissue of the training set, optimizing the loss function J_{MSE} the optimized algorithm, 1≤i≤t ; according to the step (3), randomly extracting X′_{batch} for step-1 times for continuous training, and after the training, saving the trained parameters in the Cbccon model;
wherein the Cbccon model is a convolutional neural network which consists of a plurality of the convolution layers, pool layers and a full connection layer, two filter convolution layers with 64 extracted features are used, one maximum pool layer is used to reduce the number of features, two filter convolution layers with 32 extracted features are used, one maximum pool layer is used to reduce the number of features, two filter convolution layers with 16 extracted features are used, one maximum pool layer is used to reduce the number of features, two filter convolution layers with 8 extracted features are used, one maximum pool layer is used to reduce the number of features, two filter convolution layers with 4 extracted features are used, one maximum pool layer is used to reduce the number of features, and then the data is input into a flattening layer to convert the data into one-dimensional data; finally, three full connection layers are used, in which the number of nodes is 128, 64, and the number of cell types, respectively; all convolution layers are one-dimensional, the activation function of the convolution layer is uniformly set as relu function with a step size of 1, the first two full connection layers use the relu activation function, and the last full connection layer uses the softmax layer to predict the proportion of tissue cells;
the value of the learning rate of the Cbccon model is 0.0001, the value of the testing number of times step of the model training is 5000, and the optimized algorithm of the model is set as RMSprop algorithm;
(5) using the Cbccon model trained in step (4) to predict the data, and inputtingX′ₜₑₛₜ into the trained model to obtain the prediction result, that is, the predicted tissue cell type proportion Z′ = {Z′_{1,} Z′₂,..,Zᵢ′,..,Z’ₜ} of the test set, in which Zᵢ′ is the cell proportion of a certain cell type in the tissue predicted in the test set data,1 ≤ i ≤ t .

2. The method of cell deconvolution based on the convolutional neural network according to claim 1, wherein the K is 100-5000, and the Q is 1000-100000.

3. The method of cell deconvolution based on the convolutional neural network according to claim 1, wherein using single-cell RNA sequencing data for simulation in step (1) comprises the following steps:
(1-1) determining the proportion of each cell type in a single simulated cell tissue by the formula
$${\text{Z}}_{\text{i}} = \frac{{\text{f}}_{\text{i}}}{{\sum }_{\text{i} = 1}^{\text{i=t}} {\text{f}}_{\text{i}}} ,$$
that is, determining the marking information Z {Z_{1,}Z₂,..Z_{i,..,}Zₜ} of the simulated tissue, in which Zᵢ is the cell proportion of a certain cell type in the simulated tissue; fᵢ is a random number created for a single cell type, Zᵢ has a value between [0,1], and
$${\sum }_{\text{i=1}}^{\text{i=t}} {\text{f}}_{\text{i}}$$
is the sum of random numbers created for all cell types, in which
$${\sum }_{\text{i=1}}^{\text{i=t}} {\text{Z}}_{\text{i}} = 1 , 1 \leq i \leq t ;$$
(1-2) determining the number of cells of each cell type to be actually extracted for a single simulated cell tissue by the formula Cᵢ = Zᵢ * K, that is, determining the number of cells C = {C_{1,}C₂,..,Cᵢ,..,Cₜ} extracted for each cell type of a single simulated cell tissue, in which Cᵢ is the number of cells to be extracted for a single cell type of a simulated tissue, Zᵢ is the cell proportion of a certain cell type in the simulated tissue, and K is the total number of cells in a set simulated artificial tissue, in which
$${\sum }_{\text{i=1}}^{\text{i=t}} {\text{C}}_{\text{i}} = K ,$$
and 1 ≤ i ≤ t.

4. The method of cell deconvolution based on the convolutional neural network according to claim 1, wherein the value of the batch size in step (3) is 128.
